(19) United States
(12) Patent Application Publication
(10) Pub. No.: US 2008/0183217 A1
(43) Pub. Date: Jul. 31, 2008

(54) Posterior Vertebra Locking Plate

(76) Inventor: John A. Glaser, Mt. Pleasant, SC (US)

Correspondence Address: MUSC FOUNDATION FOR RESEARCH DEVELOPMENT, 19 HAGOOD AVE, SUITE 909, P.O. BOX 250828, CHARLESTON, SC, 29425, US

(21) Appl. No.: 11/947,736

(22) Filed: Nov. 29, 2007

Related U.S. Application Data
(60) Provisional application No. 60/867,691, filed on Nov. 29, 2006.

(51) Int. Cl. A61B 17/58

(52) U.S. Cl. 606/280; 606/256

(57) ABSTRACT

Embodiments of the invention comprise a four hole locking plate designed with four locking screw holes and two lateral variable angle screw heads for posterior vertebrae fixation.

### CROSS-REFERENCE TO RELATED APPLICATIONS

This application claims the benefit of the earlier filed U.S. Provisional Patent Application entitled “Posterior Vertebra Locking Plate”, Ser. No. 60/867,691 filed on Nov. 29, 2006, said provisional application being incorporated herein by reference as if rewritten in full.

### BACKGROUND OF THE INVENTION

Cervical spinal fixation is often used to help provide stability for the spine after surgery. Fixation systems can be via wiring. In the lower cervical spine, wiring alone for fixation is not often done. Wiring is still done to fuse the upper cervical vertebral segments (C1 to C2). Wiring at this level, if the posterior cervical elements are intact, can provide a rigid construct. Although wiring can be used to fixate the C1-C2 segments in some situations, in cases of significant instability such as tumor, fracture, or rheumatoid arthritis plate fixation may be warranted. It can also be used if the posterior elements are not intact, or if a patient has already had a failed fixation with posterior wiring. Embodiments of the invention comprise a locking plate fixation device for posterior fixation of a vertebra, including a human vertebra.

### BRIEF SUMMARY OF THE INVENTION

Provided herein is a locking plate fixation device for posterior fixation of a vertebra, including a human vertebra. Embodiments of this invention allow for fixation to the first cervical vertebra using locking screw fixation. Locking screw fixation comprises placing threaded screws through threaded holes in a plate and then in to the bone.

### BRIEF DESCRIPTION OF THE DRAWINGS

FIG. 1 is an image showing C1-C2 instability where the use of embodiments of the invention may be warranted.

FIG. 2 shows a table summarizing measurements from CT scans as more fully described herein.

FIG. 3 shows two views of an embodiment of the invention, namely a four hole locking plate with four about 2.4 mm locking screw holes and two lateral variable angle screw heads.

FIG. 4 shows an embodiment of the invention affixed to C1 and C2.

FIGS. 5, 6, 7, and 8, show human cadaveric specimens

FIG. 5 shows the harvested condition.

FIG. 6 shows the destabilized spine.

FIG. 7 shows the instrumented spine conditions.

FIG. 8 shows a lateral radiograph.

FIGS. 9, 10, 11, and 12 show the mounting arrangements in testing apparatus.

FIG. 9 shows the flexion-extension test set-up.

FIG. 10 shows lateral bending set-up.

FIG. 11 shows the axial rotation test set-up.

FIG. 12 shows a close-up of the axial rotation testing arrangement.

FIGS. 13 through 36 show the results of testing.

### DETAILED DESCRIPTION OF THE INVENTION

Provided herein is a locking plate fixation device for posterior fixation of a vertebra, including a human vertebra. Embodiments can be used on the first cervical vertebra. Embodiments of the invention can be used for fixation between the first and second cervical vertebrae when surgery is indicated to fuse these two bones together. Embodiments of this invention allow for fixation to the first cervical vertebra using locking screw fixation. Locking screw fixation comprises placing threaded screws through threaded holes in a plate and then in to the bone. This locks the screws in to the plate as well as the bone. When the screws are locked into the plate, the fixation is improved.

During testing of certain embodiments of the invention, fixation of the second cervical vertebra was achieved with screws passing through the lamina. This method of fixation can avoid the vertebral artery. The method of fixation of the second cervical vertebra can be methods generally known to one of ordinary skill in the art. In certain embodiments, the screws in the second cervical vertebra are then connected to the plate on the first with metal rods secured to the heads of the screws on the second and in connectors on the plate.

### EXAMPLE 1

Current methods of C1 fixation pose surgical risk to neurologic and vascular structures. Locking plate fixation may improve the quality of fixation over wiring and decrease surgical risk. The objective was to evaluate the anatomic feasibility and biomechanical properties of a new posterior C1 locking plate in a C1-C2 fixation model.

Assessment of bony thickness of the posterior ring of C1 was performed by direct and CT measurement of fifty specimens. A locking plate was designed with four about 2.4 mm×8 mm locking screws and two variable angle screw heads to accommodate about 3.5 mm rod connection to a variable angle screw system. Five human cadaveric specimens (C0-C4) were biomechanically tested in flexion-extension, lateral bending and axial torsion. The base of the odontoid was cut to create a destabilized condition. Specimens were retested under two instrumented conditions: Locking plate fixation of C1 with translaminar screws at C2 (C1 Plate), and lateral mass screws at C1 with pars screws at C2 (Harms). Data were compared using a one-way ANOVA and SNK test.

Mean thickness measurements were larger on cadaveric specimens, suggesting that CT measurements slightly underestimate the greatest thickness. No statistically significant differences between the C1 Plate and Harms instrumented spine conditions were observed for all biomechanical tests.

Conclusion: A novel C1 posterior locking plate was designed and tested in a C1-C2 fixation model. The C1 locking plate fixation technique was biomechanically equivalent to the existing Harms technique and can be considered a viable alternative to existing fixation techniques with decreased surgical risk.

### EXAMPLE 2

This purpose of this study was to evaluate the anatomic feasibility and biomechanical properties of a new posterior locking plate of C1 in an C1-C2 fixation model. Current methods of C1 fixation include lateral mass screw fixation which places a screw near the vertebral artery and involves dissection around the C2 ganglion, and various wiring techniques. Locking plate fixation may improve the quality of fixation over wiring and decrease risk to neurologic and vascular structures.

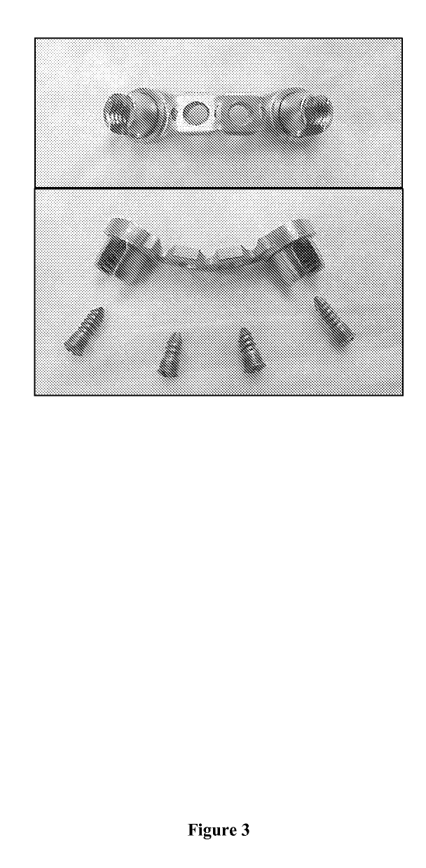

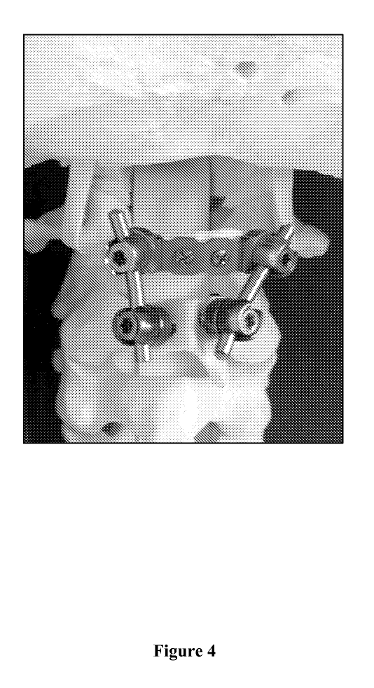

A novel locking four hole locking plate was designed with two medial 2.4 mm locking screw holes and two lateral holes to allow passage of variable angle screws. (FIG. 3). Referring to FIG. 4, using a variable angle screw system, longitudinal rod connection between the C1 locking plate and intramural screws in C2 provides rigid C1-C2 fixation.

Anatomic assessment of the posterior ring of C1 was performed to assess for appropriate screw length. 50 CT scans of were reviewed and measured at midline and 5, 10, 15 and 20 mm on each side to assess bony thickness. 50 specimens from the Haman Todd collection in Cleveland Ohio were measured at the same anatomic points. Fresh frozen human cadaveric specimens were tested. All musculature was removed, discs and ligamentous structures were retained. Instability was created by removing the base of the odontoid. Locking plate fixation of C1 with translaminar screws at C2 was compared to lateral mass screws at C1 and pars screws at C2.
Table 1 shows the results of the anatomic measurements. Mean thickness was generally larger on cadaveric specimens suggestion that CT measurements slightly underestimate the greatest thickness.

TABLE 1

| Location | | Cadaver | CT |
|---|---|---|---|
| Midline | | 8.5 (2.3-1.64) | 7.0 (0-1.25) |
| Right 5 | mm | 7.0 (3.5-1.44) | 6.0 (2.6-1.05) |
| Right 10 | mm | 6.5 (3.6-1.82) | 5.5 (3.0-8.8) |
| Right 15 | mm | 7.0 (4.6-1.18) | 6.0 (3.9-9.3) |
| Right 20 | mm | 8.8 (6.0-1.17) | 7.8 (5.7-1.12) |
| Left 5 | mm | 7.3 (4.0-1.44) | 5.8 (2.1-9.0) |
| Left 10 | mm | 6.4 (3.5-1.13) | 5.3 (2.3-7.7) |
| Left 15 | mm | 6.9 (4.4-1.02) | 5.7 (3.8-8.2) |
| Left 20 | mm | 9.2 (5.9-1.16) | 7.5 (4.8-1.0) |

All lengths are in mm, ( ) = Range

### EXAMPLE 3

### Atlantoaxial Instability

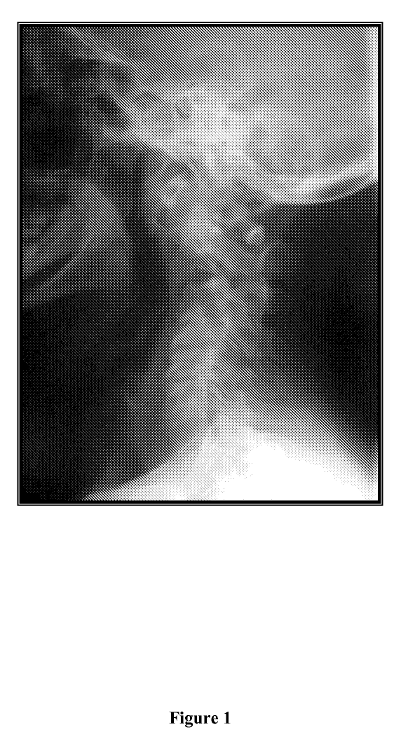

Primary stabilization of the atlantoaxial (C1-C2) joint is provided by surrounding ligamentous structures. Atlantoaxial instability, shown in FIG. 1, may arise from trauma, congenital malformation, tumor, or inflammatory disease, and is a serious progressive condition leading to pain, myelopathy or even death. Surgical intervention is often indicated to provide stability through rigid coupling of the C1 and C2 vertebra, and to decompress neural structures if required.

Commonly used fixation techniques include the Magerl-Gallie method which utilizes bilateral transarticular screws, and the Harms technique which uses polyaxial screws applied to the Lateral masses of C1 and pars of C2 connected via longitudinal rods. These methods involve placement of screws near the vertebral artery, and involve dissection around the C2 ganglion posing surgical risk to the patient. Image guided surgical techniques may be used to aid in screw placement.

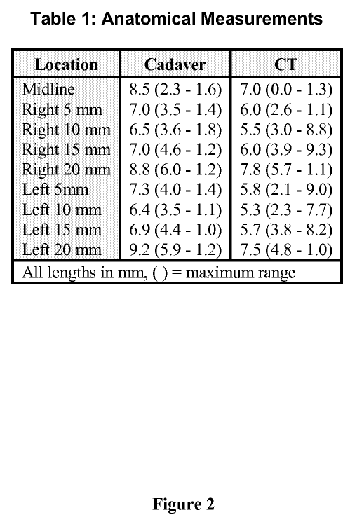

Anatomic assessment of the posterior ring of C1 was performed to assess for appropriate screw length. Fifty specimens from the Haman Todd collection in Cleveland Ohio were measured at midline and 5, 10, 15 and 20 mm on each side to assess bony thickness. Fifty CT scans were also reviewed and measured at the same anatomic points (FIG. 2). A novel four hole locking plate was designed with four 2.4 mm locking screw holes and two lateral variable angle screw heads (FIG. 3). Using a variable angle screw system, longitudinal rod connection between the C1 locking plate and intralaminar screws in C2 provides rigid C1-C2 fixation (FIG. 4).

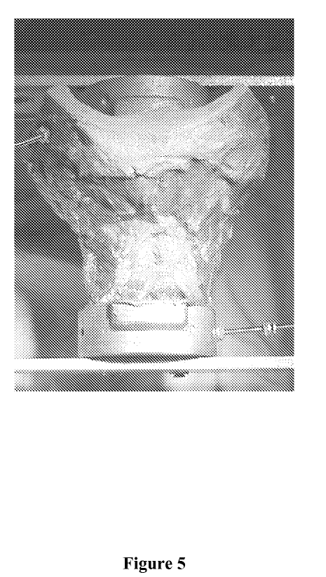

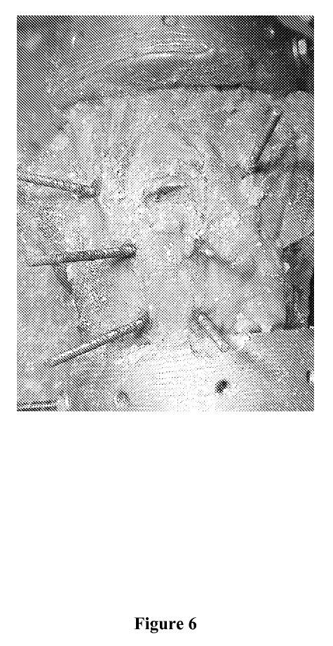

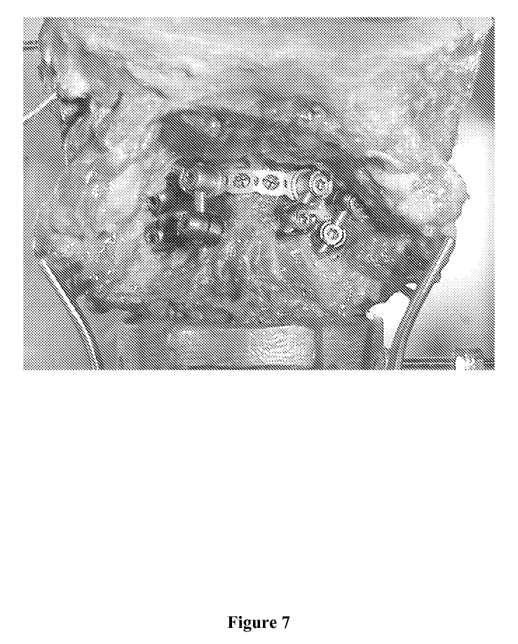

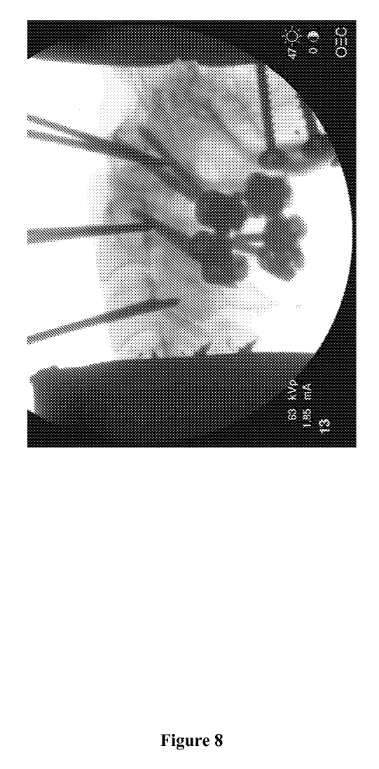

Seven fresh frozen human cadaveric specimens (C0-C4) were prepared (Mean age 76±6.7 years, four male, three female) and tested in the harvested condition (FIG. 5). The base of the odontoid was cut to create a destabilized condition (FIG. 6). Specimens were retested under two different instrumented conditions:
1. Locking plate fixation of C1 with translaminar screws at C2 (C1 Plate)
2. Lateral mass screws at C1 with pars screws at C2 (Harms).
   Four 2.4 mm×8 mm screws were used in the C1 locking plate, bicortical 3.5 mm diameter screws were used for the C1 lateral mass fixation and 3.5 mm×20 mm screws were used for all fixation at C2. All constructs used 3.5 mm rods. FIG. 8 shows a lateral radiograph view. Additional embodiments can use screws of appropriate diameter including about 2 mm to about 4 mm for the C1 locking plate and the C2 fixation, of suitable lengths. Rods of about 2 to about 4 mm can be used. Screws can include various means for affixing and binding such as threaded rods, and expansion bolts. The plate itself is configured to fit the C1 region and is angled appropriately to provide suitable placement upon or reasonable close to the C1 vertebrae. Embodiments of the plate can be curved or arced in shape to conform loosely to the general contour of C1. Alternatively, the plate can be bent or segmented with the segments being at angles to each other to form a shape that conforms loosely to the general contour of C1. One embodiment of the plate provides for a plate bent at about a 120 to 170 degree angle.

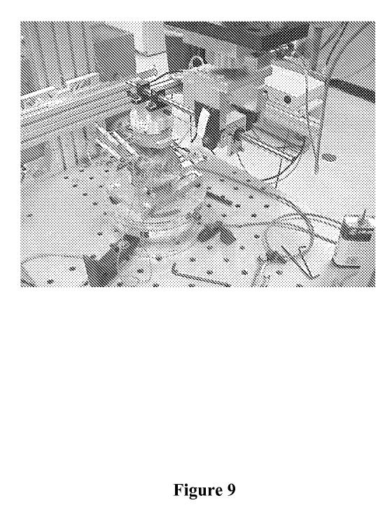

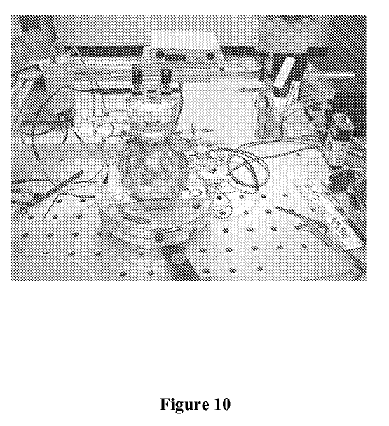

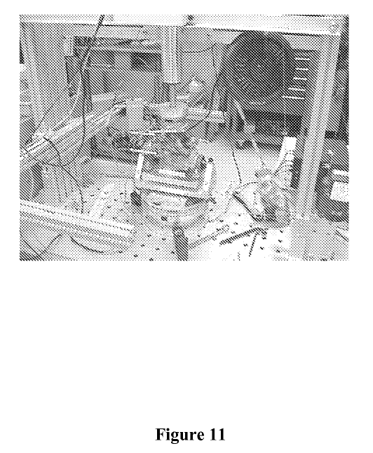

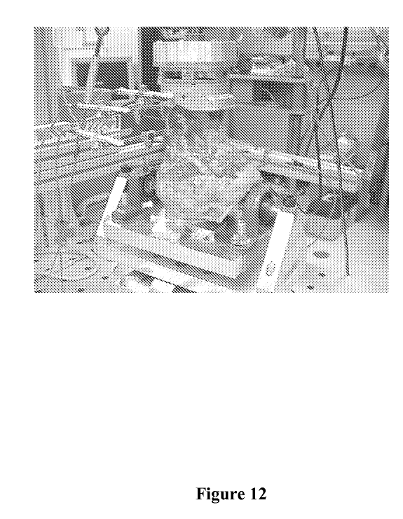

Testing Protocol: FIGS. 9-12. Test specimens were mounted in a programmable biomechanical testing frame and tested in flexion, extension, left and right lateral bending, and left and right axial rotation. For flexion, extension and lateral bending tests spines were offset 200 mm from the loading axis. For lateral bending tests, specimens were unconstrained in axial rotation; for axial rotation tests specimens were left unconstrained in lateral bending. A previously described in vitro testing protocol was adopted. Specimens were loaded with a triangular shaped displacement-time actuator waveform of 6.4 mm/s corresponding to approximately 2.0 degrees/sec overall spinal rotation. All tests proceeded until an end load limit of 1.5 Nm was reached. A non-contact real time optical measurement system was used to track segmental cervical motion for each test condition. Pairs of light emitting diodes were attached to each spinal body to measure vertebral motion.

Measurements included vertebral motions, global spinal rotation, and applied loads and moments. The total rotation and applied moment were combined to calculate the rotational flexibility of the spine. Operated level motion patterns were analyzed by normalizing the percent contribution of the C1-C2 level relative to the overall total spinal rotation (C0-C4) for the different spine conditions, with respect to the same contribution for the harvested condition. Individual MSU rotation values for each condition were statistically compared at a common end load limit. All data were compared at a common global moment end limit of 1.5 Nm. Flexibility and MSU rotation data were statistically compared using a repeated measures one-way ANOVA followed by SNK test (p<0.05). Normalized operated level data were compared using a one-way ANOVA followed by SNK test (p<0.05).

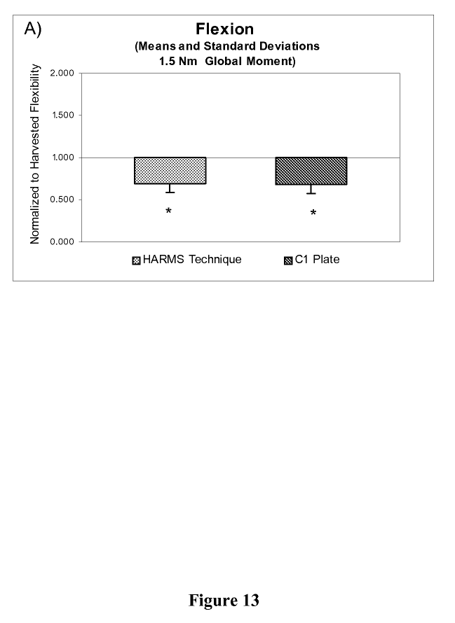

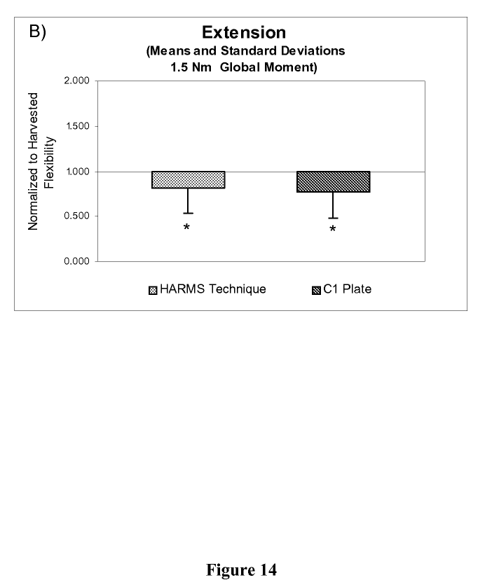

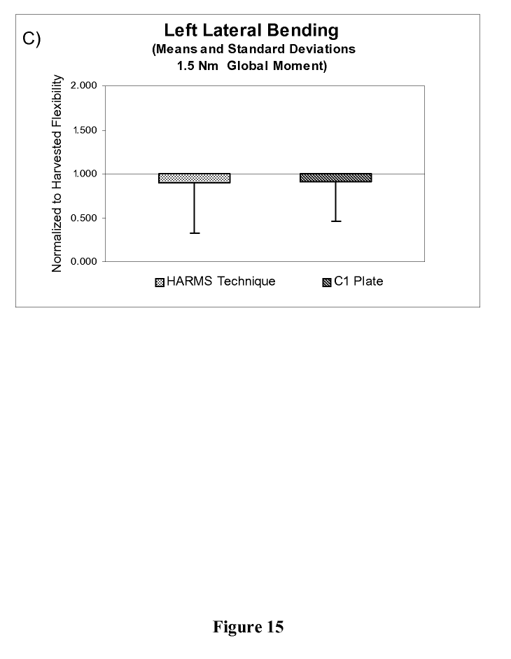

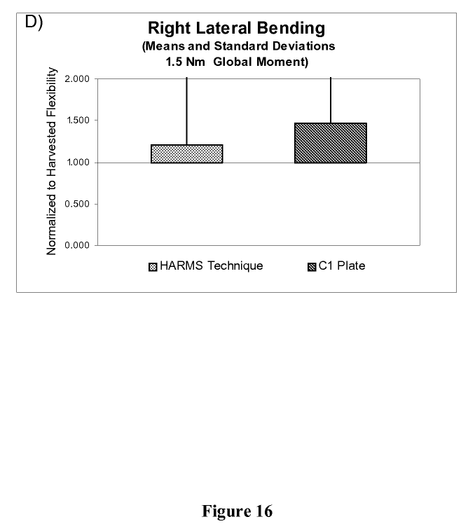

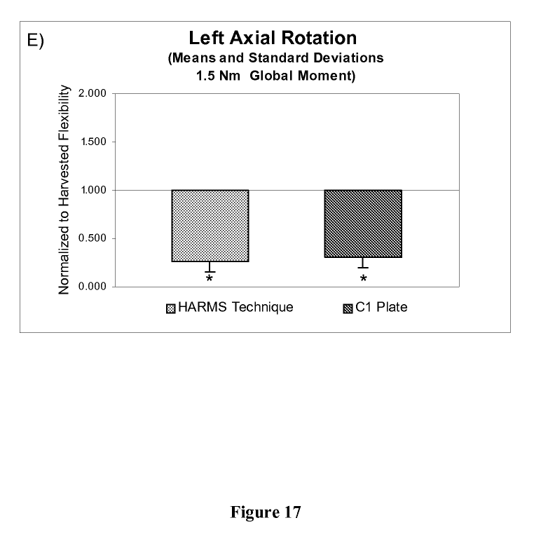

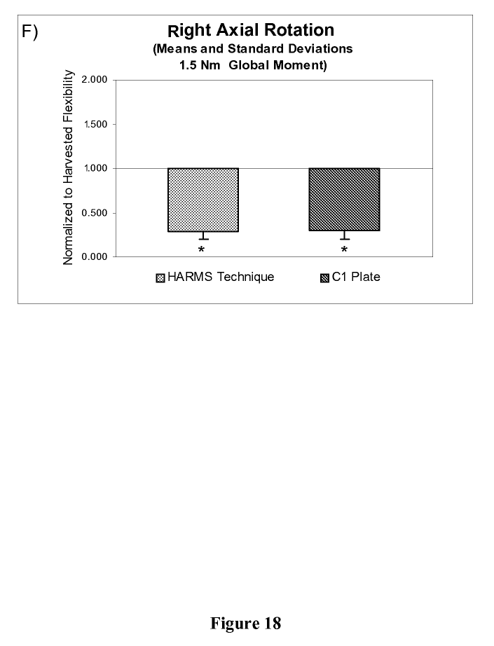

FIGS. 13 through 18 show the results of testing (Flexibility): Mean values of flexibility data for the instrumented spine conditions, shown as normalized with respect to the harvested condition in A) Flexion, B) Extension, C) Left Lateral Bending, D) Right Lateral Bending, E) Left Axial Rotation, and F) Right Axial Rotation (* Signifies significant difference with respect to the Harvested condition). Significant differences in specimen flexibility occurred between the Harms and Harvested conditions, and between the C1 Plate and Harvested conditions in flexion, extension, left and right axial rotation. No significant differences occurred between the Harms and C1 Plated conditions over all modes of loading.

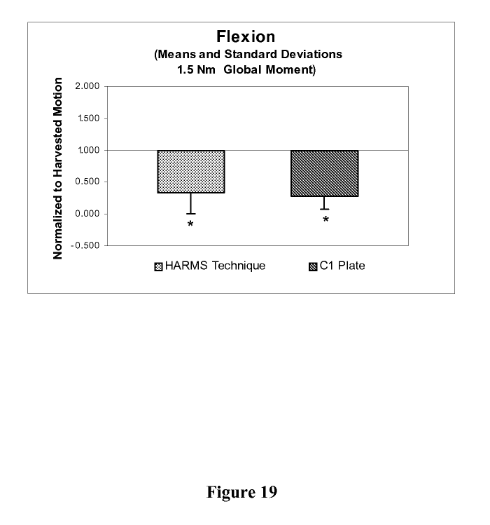

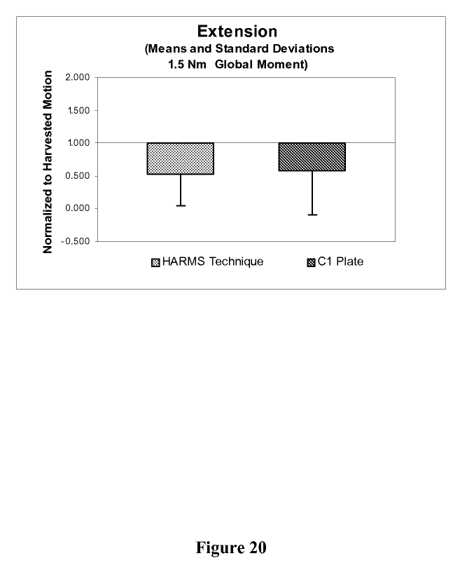

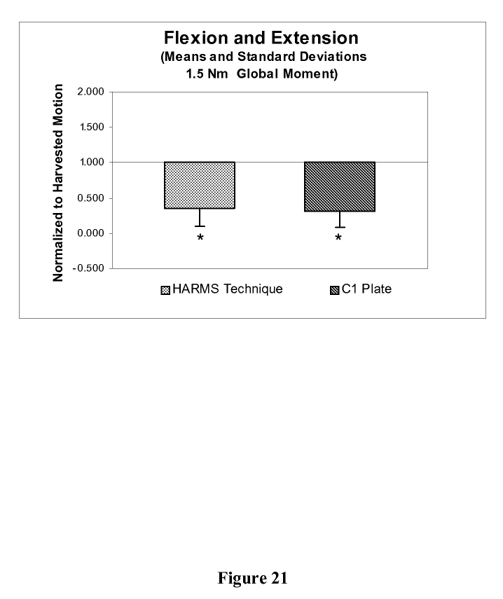

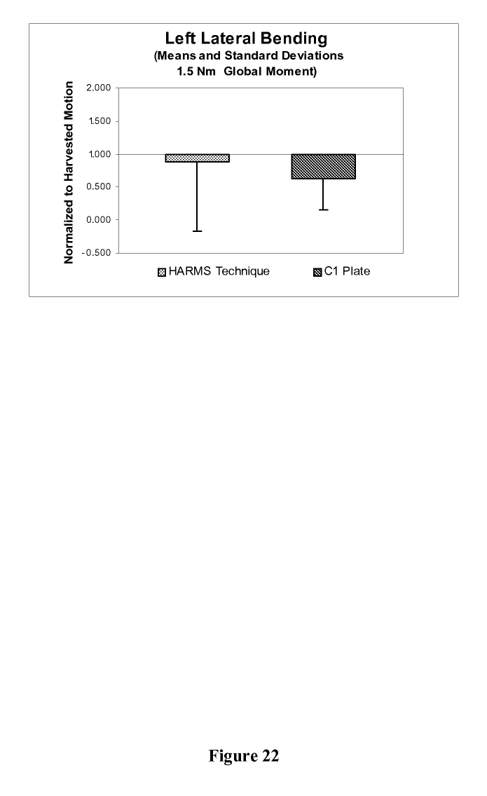

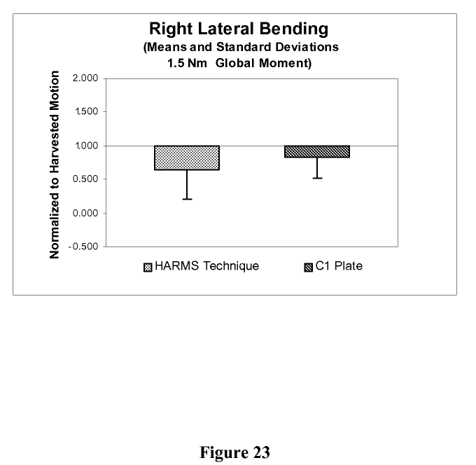

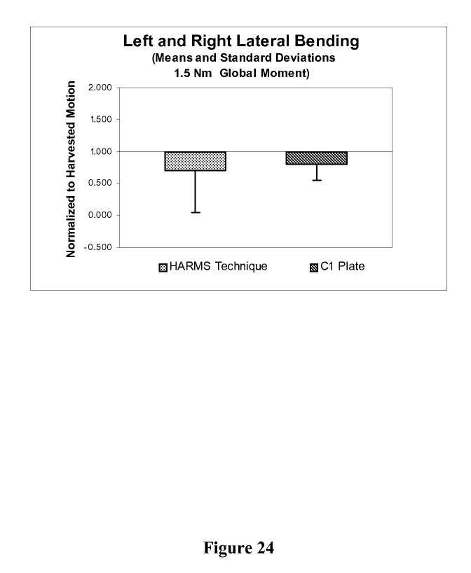

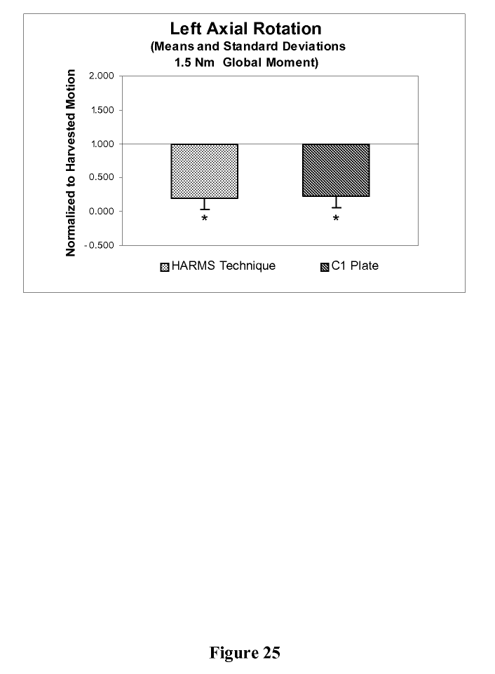

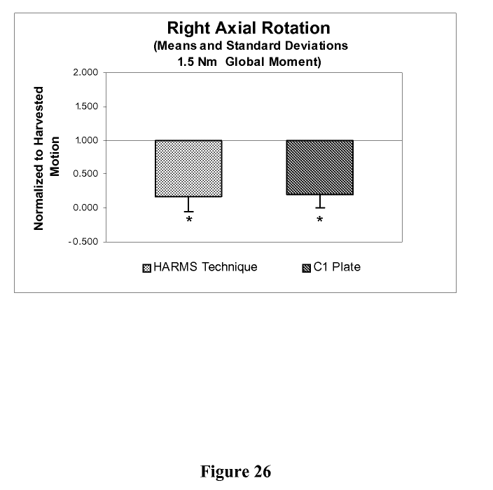

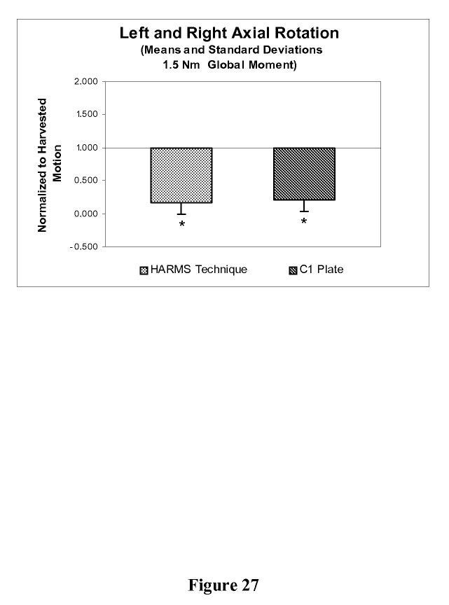

FIGS. 19 through 27 show the results of testing (Normalized Motion): Mean operated level motion of the instrumented spine conditions normalized to the harvested condition in A) Flexion and Extension, B) Left-Right Lateral Bending and C) Left-Right Axial Rotation (* Signifies significant difference with respect to Harvested condition). Significant differences occurred between the Harms and Harvested conditions, and between the C1 Plate and Harvested conditions in flexion, combined flexion and extension, and in left axial rotation, right axial rotation, and combined left and right axial rotation. No significant differences occurred between the Harms and C1 Plate condition over all modes of loading.

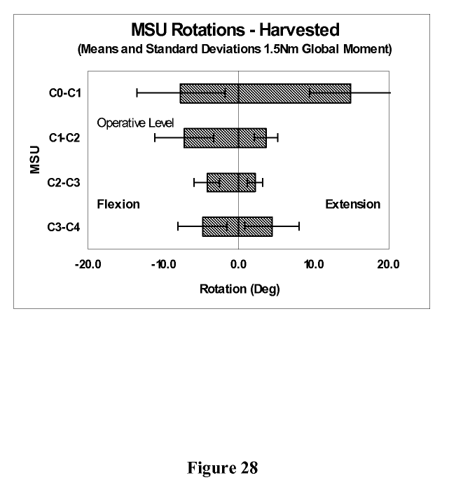

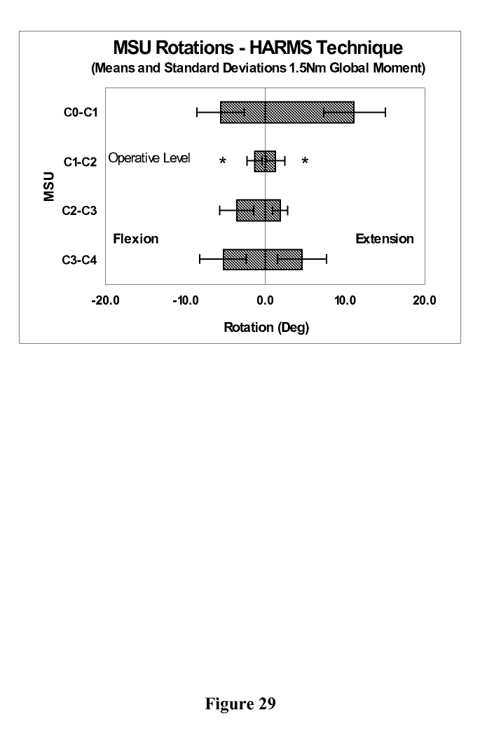

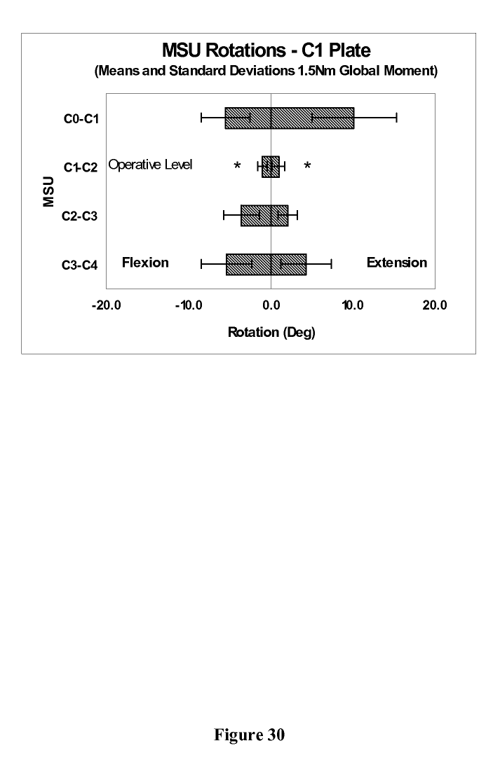

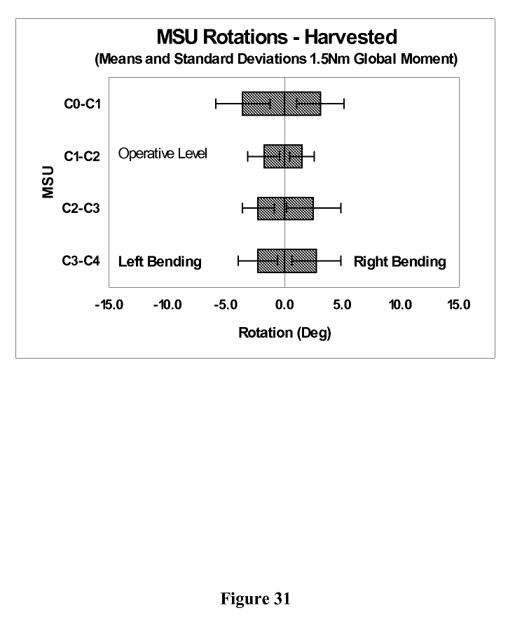

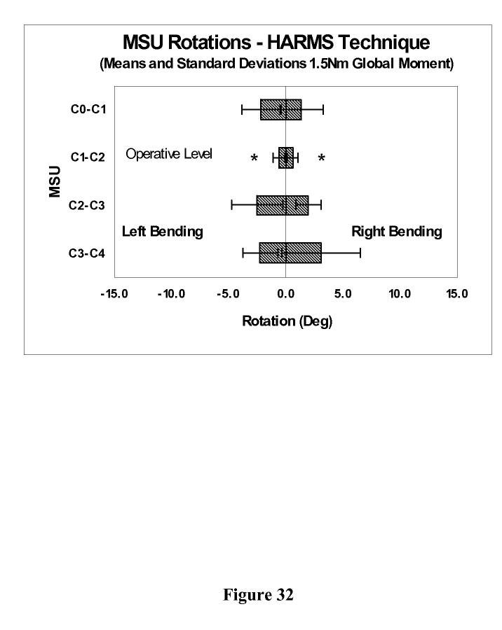

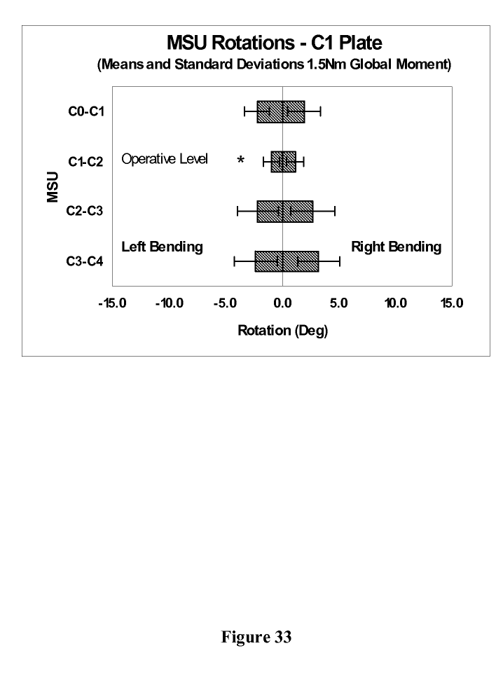

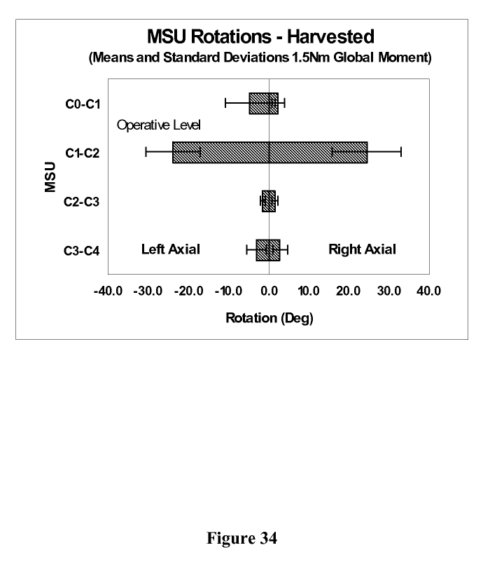

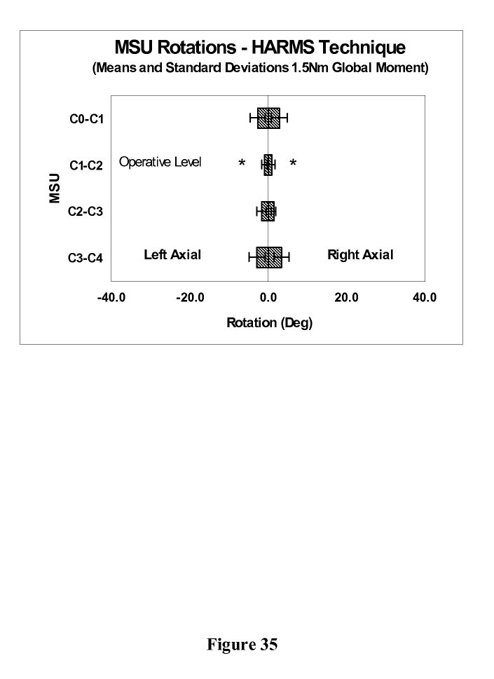

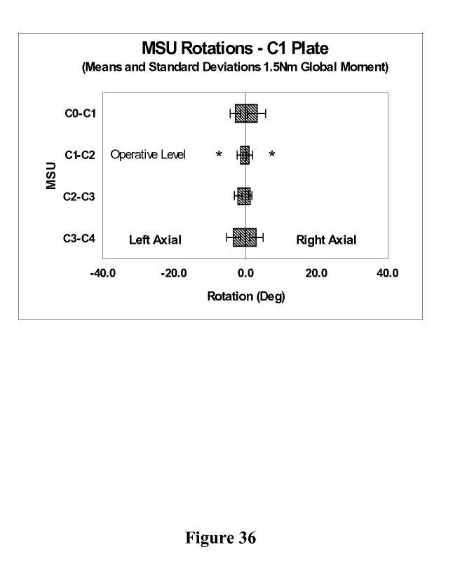

FIGS. 28 through 36 show the results of testing (Normalized Motion—Mean MSU Rotation Values): Mean MSU rotation values for harvested and instrumented spine conditions in A) Flexion and Extension, B) Left-Right Bending, C) Left-Right Axial Rotation (* Signifies significant difference with respect to Harvested condition). Significant differences in MSU rotation occurred between the Harms and Harvested conditions at the operated C1-C2 level for all modes of loading. Significant differences in MSU rotation between the C1 Plated and Harvested conditions occurred at the operated C1-C2 level for all modes of loading except right lateral bending. No significant differences between the Harms and C1 Plated conditions occurred at any MSU level.

Conclusions: A novel C1 posterior locking plate was designed and tested in a C1-C2 fixation model. No statistically significant differences between the C1 Plate and Harms instrumented spine conditions were observed for all biomechanical test conditions. The C1 locking plate fixation technique was biomechanically comparable to the existing Harms technique. The C1 locking plate can be considered a viable alternative to existing fixation techniques with decreased surgical risk.

### REFERENCES

[1] Magerl F, Stable posterior fusion of the atlas and axis by transarticular screw fixation. In Kehr P, Weidner A, eds. Cervical Spine, New York N.Y., Springer Verlag; 1986:322-7.
[2] Harms J, Melcher R P, Posterior C1-C2 fusion with polyaxial screw and rod fixation. Spine 2001; 26:2467-71.
[3] Thomas A, DiAngelo D J, Kelly B P, Design of a portable biomechanical testing system to study tissue-implant mechanics. Third Tennessee Conference on Biomedical Engineering, April, 2000.
[4] DiAngelo D J, Foley K T, Schwab J S, Morrow B R et al, In vitro biomechanics of cervical disc arthroplasty with the ProDisc-C total disc implant, Neurosurgical Focus, September, 2004.
[5] DiAngelo D J, Foley K T, An improved biomechanical testing protocol for evaluating multilevel instrumentation in a human cadaveric corpectomy model, in Spinal Implants: Are we evaluating them appropriately?, ASTM STP1431, Melkerson M N, Griffith S L, Kirkpatrick J S, eds., American society for standards and materials, West Conshohocken, Pa., 155-172, 2003.

I claim:

1. A vertebrae fixation device comprising a plate wherein said plate comprises four locking screw holes and two lateral variable angle screw heads.

2. The device of claim 1 further comprising rods that connect with said variable angle screw heads wherein said rods can connect with an additional fixation device attached to a second vertebrae.
